(19) United States
(12) Patent Application Publication
(10) Pub. No.: US 2009/0136623 A1
(43) Pub. Date: May 28, 2009

(54) GROWTH INHIBITORY COMPOSITION AGAINST PATHOGENIC BACTERIA OF MEAT BASED FOOD STUFF COMPRISING IGY

(76) Inventor: Min-Seok SONG, Seoul (KR)

Correspondence Address: BIRCH STEWART KOLASCH & BIRCH, PO BOX 747, FALLS CHURCH, VA, 22040-0747, US

(21) Appl. No.: 12/282,402

(22) PCT Filed: Mar. 12, 2007
(86) PCT No.: PCT/KR2007/001198

§ 371 Date: Sep. 10, 2008

(30) Foreign Application Priority Data
Mar. 10, 2006 (KR) 10-2006-0022834

(51) Int. Cl. A23B 4/20; A61K 39/02; A61K 39/07; A61K 39/08; A61K 39/085; C07K 16/18

(52) U.S. Cl. 426/61; 424/234.1; 424/246.1; 424/247.1; 424/243.1; 424/258.1; 530/387.1

(57) ABSTRACT

The present invention relates to growth-inhibitory composition against pathogenic bacteria of meat based food stuff comprising IgY, a specific immunoglobulin derived from yolk of egg. The 12 representative bacteria and other microbes deteriorating quality of process meat products that the IgY of the present invention targets include Aeromonas hydrophila, Bacilluis cereus, Campylobacter jejuni, Clostridium perfringens, 0157: H7 (Escherichia coli 0157:H7), Lactobacillus, Listeria monocytogens, Sacromyces cerevisae, Salmonella enteritidis, Salmonella typhimurium, Staphylococcus aureus, and Staphylococcus epidermidis. As the form of antigen, the 12 bacteria are injected to chickens separately to obtain IgY of the present invention against the 12 pathogens.

### TECHNICAL FIELD

The present invention relates to a method for manufacturing food additives which curbs the growth of pathogenic bacteria of meat based food stuff comprising IgY (immunoglobulin in yolk) and a use of the food additives.

### BACKGROUND ART

In order to improve the quality of processed meat food stuff, such as ham, sausage and broiled meat, core technologies in a wide range of areas are required with respect to the fact that these food products come from different sources of meat like pork, mutton, beef and chicken. The technologies that play significant roles in improving overall characteristics of the processed meat food stuff include diverse technologies such as a taste enhancing technology based on using a new spice, a cost reduction technology based on using protein-based materials with higher water holding capacity, a texture and flavor making technology based on adding enzymes or protein-based materials, and a non-thermal sterilization technology that can minimize quality deterioration caused by heat. Of these, the non-thermal sterilization technology sterilizing meat food stuff by minimizing heat treatment is the most crucial to remarkably improve quality of meat-based food stuff. Two types of treatment technologies currently under development include the chemical treatment technology which uses natural anti-microbe substances and the physical treatment technology that uses an electric field, a magnetic field, extra high-pressure or an optical energy. With respect to both the current level of technologies and volatile production cost, the non-thermal sterilization technology that uses natural anti-bacterial substances is more advantage to be applied to the food industry.

Various natural anti-microbial substances including bacteriocine extracts, Chitosan medicine, lysozyme medicine, calcium medicine, various organic acids, grapefruit seed extract, polylysine, and vitamin BI derivative have been developed and are available in the market. 2%˜3% of organic acid applications like Potassium lactate and Calcium lactate are being used to ensure the safety of processed meat food stuff from bacteria. However, most commercial anti-bacterial substances have the inactivation mechanism that surface active agents harm the bacteria's cell membrane on the hydrophobic and hydrophilic areas of bacteria's cells, resulting in making germs extinct, but the problem lies on the fact that the anti-bacterial effect of the surface active agents is significantly impeded on protein-based products like processed meat products that contains a lot of polar groups. Precisely, since polar groups including protein in the food stuff previously keep surface active agents of the anti-bacterial substances from effectively reaching and being combined in the bacteria's cell membrane, effective sterilization is not carried out. Due to the critical defect that commercial anti-bacterial substances that show excellent sterilization or growth-inhibitory effects on the medium actually encounter a significantly reduced effect in real food, particularly in protein-based food stuff, anti-bacterial substances that can be used in meat-based processed food stuff has been very restricted except for natural anti-bacterial substances from the organic acid.

Therefore, it is the most essential to develop and secure natural anti-bacterial substances with effective sterilization and bacterial growth-inhibitory functions that can be used for processed meat products mainly composed of proteins as their main ingredients. This will also be the basis for non-thermal sterilization, because developing and securing effective natural anti-bacterial substances will be a differentiated technology in minimizing quality deterioration and the decline in yield caused by secondary thermal treatment sterilization.

With respect to the aforementioned aspects, the present invention has been completed through developing growth inhibitory composition against pathogenic bacteria of meat based food stuff comprising IgY that has a different anti-bacterial mechanism and can be used as an effective natural preservative for meat-based processed food stuff, and observing and analyzing the growth inhibitory effect of the invented composition when applied to processed meat food stuff.

IgY is an antibody, a type of special immunoglobulin, which is passed from the mother chicken to the embryo via the egg yolk. It is completely different from natural anti-bacterial substances with surface active agents in terms of production method and functional mechanism. To produce IgY, targeted pathogenic bacteria for sterilization or growth inhibition, such as Helicobacter, E. coli, Staphyfococcus aureus, and Salmonella, are injected into the mother chicken to generate a special immunoglobulin (antibody) in the egg yolk that can defend against the targeted bacteria. The special immunoglobulin accumulated in the egg yolk is referred to as IgY, and unlike ordinary antibiotics it is characterized that IgY has no side-effect to human body as well as no tolerance to bacteria.

So far, IgY has been mainly used only in functional eggs, dairy products, soybean milk, functional feed and functional cosmetic products. There is no previous case wherein IgY was used in meat-based products as suggested in the present invention. No attempt was previously made to use IgY for the purpose of sterilization and growth inhibition of various pathogenic bacteria that could negatively. impact on the safety and quality of meat-based products, or to manufacture IgY as a powder- or liquid-type substance. Existing cases of research and development featured in patents and research literature both in Korea and overseas are as follows: Korean Patent Registration No. 0437500 relates to a food additive by taking two bacteria that cause mouth diseases, Streptococcus mutants and Actinomyces israelii, as antigens, and injecting them to different chickens separately to produce IgY against each of the relevant pathogenic bacteria. In addition, the same patent also relates to functional ice cream with the specific food additive. Korean Patent Registration No. 0415911 relates to how to produce eggs that can prevent enteritis by simultaneously injecting E. coli which causes enteritis and gastritis and Helicobacter pylori which causes enteritis into a young chicken. In addition, Korean Patent Registration No. 0406870 suggests how to separate and purify IgY from the water soluble ingredient of the yolk. Korean Patent Registration No. 0392564 introduces how to produce functional eggs that have specific immunoglobulin against Propionibacterium acnes that causes acne. Korean Patent Application No. 2001-0046634 relates to functional soybean milk and yoghurt with liquid yolk containing IgY against Helicobacter. Most research literature and studies conducted in this area describe how to produce the IgY antibody from egg yolk and features of IgY, and studies for sterilization and growth inhibition have been conducted using a few specific bacteria such as Helicobacter, E. coli and Salmonella.

The motivation behind the invention—as being discussed in this paper—is based on the facts that existing studies, development and commercialization of IgY were heavily focused on functional materials and foods, and that there are no commercially applicable and effective natural anti-bacterial substances for processed meat products. By inventing a commercially-available natural anti-bacterial substance that has selective antibiotic effects against the targeted bacteria, we could develop and complete a progressive non-thermal sterilization technology that enables companies to commercialize high-quality products in various forms such as sterilized packaged products without any need of additional thermal treatment after packaging and the products packed in the tray immediately after cooking.

### DISCLOSURE

### Technical Problem

The object of the present invention is to provide growth inhibitory composition against pathogenic bacteria of meat based food stuff comprising IgY.

Another object of the present invention is to provide a method of sterilizing and inhibiting the growth of the pathogenic bacteria in the meat-based processed food stuff by treating the aforementioned composition.

### Technical Solution

The present invention is composed of several stages as follows: the stage wherein yolk containing IgY is produced; the stage wherein IgY is separated from the yolk and purified; the stage wherein liquid- and powder-type yolk containing IgY is manufactured; the stage wherein stability of IgY obtained in the previous stage is inspected; the stage wherein the antibacterial effects of the powder- and liquid-type yolk containing IgY is measured and evaluated.

The present invention provides a method for manufacturing IgY comprising the steps:

i) producing egg containing specific immunoglobulin by injecting vaccines against pathogenic bacteria and other microbes that deteriorate product quality to chickens;

ii) sterilizing the yolk separated from the egg at a low temperature; and

iii) adding acidic water solution to the sterilized yolk and centrifuging the resulted mixture.

In Step ii), the low temperature sterilization is performed at 50-75° C. for 15 minutes to an hour. If the sterilization temperature is lower than 50° C., the sterilization is not sufficiently done. If the temperature is higher than 75° C., then the yolk will be denaturalized by heat. If the sterilization duration is shorter than 15 minutes, then sterilization is insufficiently done whereas it is not necessary to perform the sterilization longer than an hour because no greater sterilization effect will occur after an hour of sterilization.

In Step iii), it is characterized that centrifugation is performed after the final acidity is adjusted to between pH 5.2 and pH 5.5 by adding acidic water solution to the sterilized yolk.

In addition, the present invention also provides yolk that contains immunoglobulin specifically manufactured through two steps as follows: the Step 1 wherein eggs with specific immunoglobulin are produced by injecting to chickens vaccines of pathogenic bacteria and other bacteria that deteriorate product quality and the Step ii) wherein the yolk separated from the aforementioned eggs is sterilized at a low temperature.

The yolk sterilized at a low temperature can be lyophilized—or freeze dried—in order to convert it into powder-type yolk containing IgY.

Furthermore, the invention provides food additive composition with sterilization or decay repressive functions for meat-based food stuff that contains one or more of following groups: the abovementioned IgY, the egg yolk containing the abovementioned IgY, and the egg yolk powder containing the above mentioned IgY.

### DESCRIPTION OF DRAWINGS

FIG. 1 shows the manufacturing process of IgY of the present invention.

FIG. 2 shows the graph of anti-bacterial activity titer of IgY of the present invention.

FIG. 3 shows the graph that indicates acidity and temperature stability of IgY of the present invention.

FIG. 4 shows the graph that indicates sterilization and growth-inhibitory effects of egg yolk powder with IgY at the temperature of 2000 per storage period of Grill Winner sausage.

FIG. 5 illustrates the mechanism of Salmonella cell membrane damage by IgY of the present invention.

FIG. 6 is a photograph that shows sterilization and growth-inhibitory effects of IgY of the present invention at the temperature of 2000 per storage period of Grill Winner sausage. Here, “A” is the control group and “B” is the experimental group treated with IgY.

FIG. 7 is a graph that indicates sterilization effect against 0157:H7 (Escherichia coli 0157:H7) by IgY of the present invention.

FIG. 8 is a graph that indicates the sterilization effect against Salmonella enteritidis by IgY of the present invention.

FIG. 9 is a graph that indicates the sterilization effect against Salmonella typhimurium by IgY of the present invention.

FIG. 10 is what was observed through the electron microscope that indicates cell membrane damaging mechanism of 0157:H7 (Escherichia coli 0157:H7) by IgY of the present invention.

FIG. 11 is what was observed on the Grill Winner sausage wherein liquid-type IgY of the present invention was treated. The observation was made after the IgY-treated sausage was kept for seven (7) days at the temperature of 37° C. Here, “A” is the control group, and “B” is the experimental group treated with IgY.

### MODE FOR INVENTION

In the present invention, the term, “IgY”—or Immunoglobulin in Yolk—means specific immunoglobulin (antibody) that is passed from the mother chicken to the embryo via the egg yolk. All types of IgY composition, such as liquid- or powder-type substances, are included in the terminology of IgY in the present invention.

There are about 20 types of pathogenic bacteria and other microbes that contribute to deterioration of quality in processed meat products. These include Aeronomas hydrophila, Bacillus cereus, Campylobacter jejuni, Clostridium perfingens, 0517:H7 (Escherichia coli 0157:H7), Lactobacillius, Listeria monocytogens, Sacromyces cerevisae, Salmonella enteritidis, Salmonella typhimurium, Staphylococcus aureus, Staphylococcus epidermidis, Clostridium botulinum, Vibrio spp and Penicillium.

Of the 20 bacteria above, the present invention only selected 12 pathogenic bacteria as final targets that include gram negative and positive bacteria and heat-resistant “sporeformers.” These bacteria have potential risks of causing decomposition, gas generation, phlegmatic substance formation, and food poisoning toxin formation in products with relatively strong thermal treatment as well as those with weak thermal treatment and products packed on the aseptic tray without post sterilization.

The pathogenic bacteria and microbes discussed in the present invention that deteriorate product quality include 0157:H7 (Escherichia coli 0157:H7, Gram-negative), Salmonella entiritidis, Salmonella typhimurium, Campylobacter jejuni, Staphylococcus aureus (Gram-positive), Staphylococcus epidermidis, Listeria monocytogens, Bacillus cereus (Gram-positive and heat-resistant sporeformer), Clostridium perfringens, and Sacromyces cerevisae which is one of the representative bacteria responsible for causing the deterioration of product quality. the present invention can provide information on IgY—or yolk containing IgY with excellent antibacterial activity—against the abovementioned targets known as the pathogenic bacteria and other microbes that cause product quality deterioration.

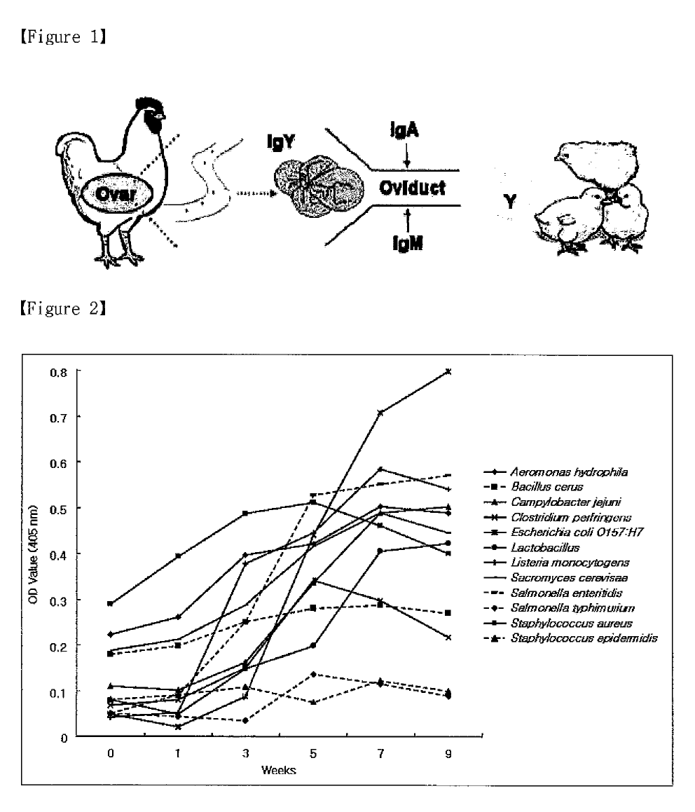

In the present invention, the selected target bacteria were injected into the mother chickens, and IgY (specific antibodies that can block the injected 12 bacteria) was primarily obtained from the yolk of the eggs laid by the mother chickens. The chemical composition of the product has the highest content of protein, followed by lipid, ash and water. The total content of IgY antibodies against the 12 pathogenic microbes amounts to 15.5 mg/g (FIG. 1). In order to directly verify the IgY's sterilizing and growth-inhibitory effects against the 12 bacteria, ELISA (Enzyme-Linked Immunosorbent Assay), an indirect enzyme immune response method, was used to observe changes in Optical Density (OD) value in the course of the antigen-antibody response. The increase in OD value indicates that IgY's selective response to the specific pathogen is high. Therefore, this can serve as an indirect index indicating high sterilizing and growth-inhibitory effects. The test results represent that IgY of the present invention showed high OD values in most of the 12 pathogens, except for Staphylococcus epidermidis and Salmonella typhimurium—which means that the IgY can inhibit growth of the specific pathogens. The inactivation mechanism of IgY can be understood as a response by the antibody binding to the cell membrane. In detail, IgY is not a monoclonal but a polyclonal antibody, whose amount increases during the six-hour culture period with bacteria, and it shows specific bactericidal function that curbs growth of the bacteria. The effect to restrain proliferation of bacteria for a certain period of time is generated through the mechanism wherein the two sites of the Fab fragments of IgY bind to ouster membrane protein (fimbriae) or lipopolysaccharide of the bacteria to create agglutination effect and therefore damage the function of the cell membrane.

To manufacture liquid-type yolk containing IgY, the egg containing IgY whose antibacterial effect was indirectly verified through ELISA is grinded to separate yolk. Then the yolk is stirred at a low-speed while sterilized for 15 to 60 minutes at the temperature of 50˜75° C. at the same time. Through this method, the liquid-type yolk containing IgY in aseptic condition using low temperature sterilization can be obtained.

In addition, If the yolk containing IgY that is sterilized at a low temperature is cooled down overnight at a cooling room with temperature of 1˜10° C. and then spray-dried in the aseptic condition, then powder-type yolk containing IgY can be obtained.

Furthermore, IgY water solution can be also manufactured through the following methods: the separated yolk is sterilized at a low temperature, and diluted six to nine fold with acid treated water solution of between pH 2 and pH 5 to produce the final acidity of between pH 5.2 and 5.5. Afterwards, the yolk solution is stored in the cooling condition of 1˜10° C. for at least 6 hours (preferably between 6 and 24 hours), and is centrifuged for 15 to 60 minutes at the speed of 3000-6000 rpm. In this way, the IgY in the supernatant and the lipo protein in the pellet can be separated to obtain IgY water solution. In addition, through lyophilization (freeze-drying) of the IgY water solution, powder-type IgY can be also obtained. Powder-type substance is more useful in many ways than liquid-type substance with respect to convenience in storage and distribution.

The IgY separation method explained above is efficient, as more than 90% of antibody activity titer can be secured with the simple process using only a centrifuge (centrifugator) and lyophilizer as the required equipment.

The bacteria sterilization and growth-inhibitory effects of IgY manufactured by the process was observed after the IgY was applied to the actual processed meat product. Each of IgY-mixed powder and liquid with antibacterial effect against 12 pathogens was separately applied to Grill Winner sausages (Brand name, CJ CORP., Republic of Korea) selected as model food. In the course of manufacturing the sausages, IgY was added at the rate of 0.5% of the weight of the sausages. The sausages were observed at 10° C., 15° C., 20° C., and 35° C. for eight (8) days up to fifty-six (56) days to analyze the total number of bacteria, sensory tests and gas generation that indicate decomposition. In the analysis, results showed sterilization and growth-inhibitory effects including no gas generation from bacteria caused by 10¹ of microbial sterilization and growth inhibition of bacteria. As IgY never affects the taste of processed food stuff on top of its effective antimicrobial effect, the IgY has a significant advantage that is expected to overcome the limited use of the existing commercial natural antimicrobial substances.

In another aspect, the present invention relates to the method to sterilize and inhibit the growth of pathogens and other microbes that contribute to the deterioration of meat-based food stuff by treating it with the above-mentioned composition.

At this point, the composition shall be used within the effective amount range. While the treatment method is not specified, the composition can be added to the meat-based food stuff in the course of the food manufacturing.

The present invention will be described in detail herein below with reference to experimental examples. It should be also clearly understood that the foregoing description is made only by way of examples, and the present invention is not, therefore, limited only to the particular embodiment.

### EXAMPLE 1

### Manufacturing Yolk Containing IgY and its Composition Through Separation and Purification

Powder- and liquid-type yolk containing IgY was manufactured through the following process (FIG. 1). As for the overall manufacturing process, 12 pathogens that cause problems in the aspects of hygiene and quality of meat-based food stuff were selected, and each of the bacteria vaccines was injected to randomly sampled mother chickens to obtain IgY substances specific to the bacteria. The IgY is separated and purified to measure its activity. When the effective antibacterial function is indirectly proven, the yolk containing IgY is manufactured in either liquid- or powder-type through low temperature sterilization and drying process. Detailed explanation of the present invention by process is as follows:

Process 1: Producing Eggs Containing IgY

For desirable execution of the present invention, vaccines against 12 types of target bacteria including Bacillus, Clostridium, Salmonella and E. coli were injected to single comb white leghorn chickens—between 19 and 21 weeks old—to obtain 12 types of eggs that contain IgY against 12 types of bacteria, respectively. The injected bacteria vaccines are inactivated bacteria with denaturalized cell membranes by way of thermal or chemical treatment, which were mixed with immune-stimulating substances and injected through the chest muscle of the chickens. In order to generate the maximum amount of antibodies, the vaccines were injected at least twice. In the present invention, the memory B cells were activated after the primary vaccine injection, and then a second injection was conducted to cause cell divisions by geometric progression to produce a large amount of specific antibodies.

Process 2: Separating and Purifying IgY

In the present invention, IgY was separated from the yolk of the eggs, and its antibacterial activity was indirectly measured through ELISA, an immune enzyme response method, to determine whether the yolk containing IgY is suitable or not.

It is very important to efficiently separate IgY, a water soluble ingredient, from the liquid ingredients of the yolk. Therefore, in the present invention, the yolk was mixed with acidic saline solution (pH 5.0˜pH 5.5) nine times and after six hours, low density lipoproteins were precipitated in the mixture so that only the water soluble substance, IgY, could be separated through centrifugation.

The separated IgY was then lyophilized. Through ELISA (OD value at 450 nm), which is an indirect index of antibacterial effect, specific activities of the IgY against specific pathogens were analyzed using OD value. A high OD value indirectly indicates that it has a high antibacterial effect against the pathogen. The results are set forth in FIG. 2. The results reveal effective antibacterial effect of IgY against pathogens and other bacteria deteriorating product quality. Of particular note, the antibacterial effect was especially excellent against seven (7) pathogens such as 0157:H7 (Escherichia coli 0157:H7) and Salmonella enteritidis (Gram-negative), Staphylococcus aureus (Gram-positive), Listeria monocytogens, Bacillus cereus (Gram-positive and Heat-resistant sporeformer), Clostridium perfringens, and Sacromyces cerevisae that is the representative quality deteriorating microbe.

According to the results of this Implementation Example, the IgY composition showed effective 0V values against most of the 12 pathogens, except for Staphylococcus epidermidis and Salmonella typhimurium which indicates its excellent performance to inhibit growth of the specific pathogens (refer to Table 1).

TABLE 1
Biochemical characteristics of yolk containing
IgY of the present invention

| Composition | Content (%) |
|---|---|
| Moisture | 3.2% |
| Protein | 49.1% |
| Fat | 42.3% |
| Carbohydrate | 0.9% |
| Ash | 4.5% |
| Total IgY | 15.5 mg/g |
| Specific IgY | Titer (OD value: |
| | absorbance at 405 nm) |
| Aeromonas hydrophila | 0.502 |
| Bacillus cerus | 0.287 |
| Campylobacter jejuni | 0.488 |
| Clostridium perfringens | 0.297 |
| O157:H7 (Escherichia coli O157:H7) | 0.707 |
| Lactobacillus | 0.404 |
| Listeria monocytogens | 0.584 |
| Sacromyces cerevisae | 0.489 |
| Salmonella enteritidis | 0.552 |
| Salmonella typhimurium | 0.115 |
| Staphylococcus aureus | 0.461 |
| Staphylococcus epidermidis | 0.122 |

Process 3: Manufacturing Liquid- and Powder-Type Yolk Containing IgY

As the analysis of antibacterial activity of the IgY in the yolk of the present invention indicated effective values, the next stage proceeded to manufacture liquid- and powder-type yolk containing IgY. The eggs containing IgY whose antibacterial effect was indirectly verified through the above explained ELISA method was grinded to separate the yolk. The yolk was then stirred at a low speed while simultaneously sterilized at the low temperature of 62° C. for 30 minutes. As a next step, the liquid-type yolk was cooled down overnight in a cooling room at the temperature of 4° C. and then spray-dried in the aseptic condition to be manufactured into powder-type yolk. The yield of the powder manufacturing process was 83% and IgY activity in the powder-type yolk was 90.7%, which can be interpreted that the considerably high value was maintained after the processes.

The results of the experiment on the composition manufactured in accordance with the Implementation Example are as follows.

### EXPERIMENTAL EXAMPLE 1

### Measurement of IgY Stability

In order to commercialize the IgY of the present invention as a commercial natural antibacterial substance, it is essential to secure the stability of IgY activity in various conditions. Therefore, as a result of the observation of stability at the pH and temperature set forth in the above Example, it was confirmed that the function was lost at pH 3 or below due to denaturalization, and the antibody function was reduced at pH 9 or above. As the acidity of processed foods generally ranges from pH 4 to pH 7, it was evaluated that the activity of IgY is well maintained in this range.

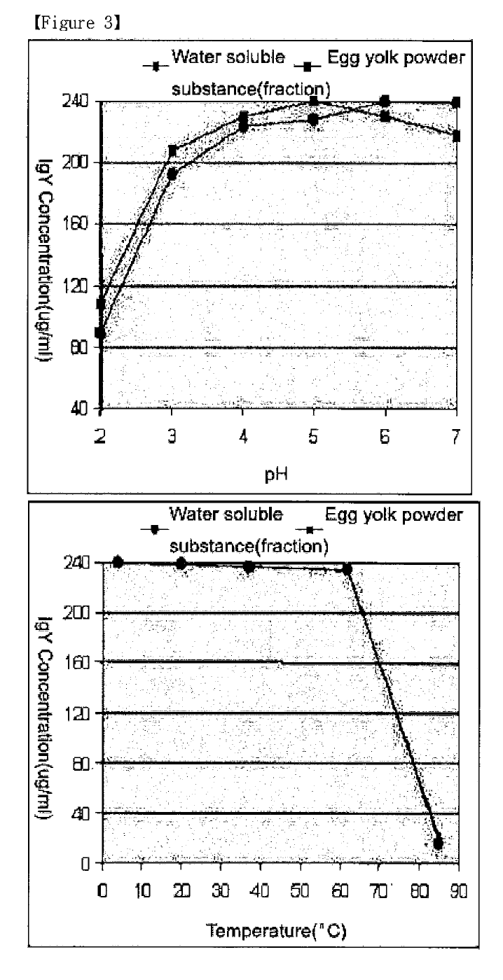

In general, in order to use egg derivative materials in food, pasteurization is performed at the temperature of 65° C. for 5 minutes. In this condition, IgY showed 99% of antibacterial activity. However, it lost antibacterial activity by more than 60% when heated at the temperature of 75° C. or higher for one hour. Therefore, it was concluded that this substance was suitable for non-thermal sterilization products (FIG. 3).

### EXPERIMENTAL EXAMPLE 2

### Measurement of Antibacterial Effect of Powder-Type Yolk Containing 12 Types of IgY

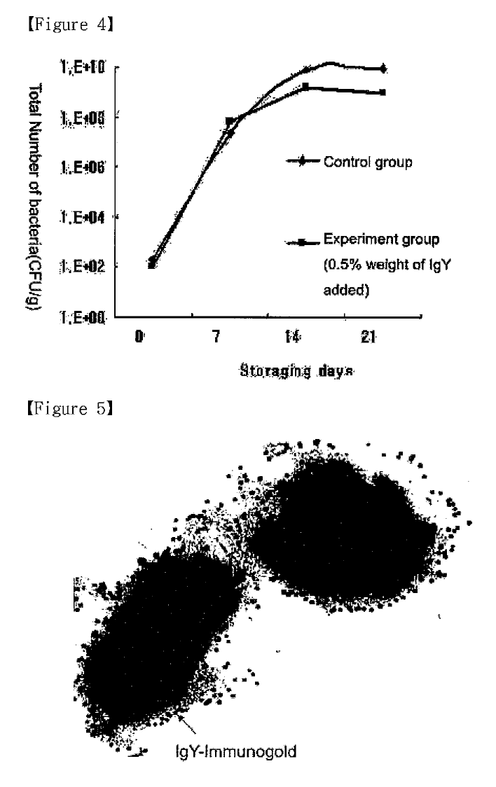

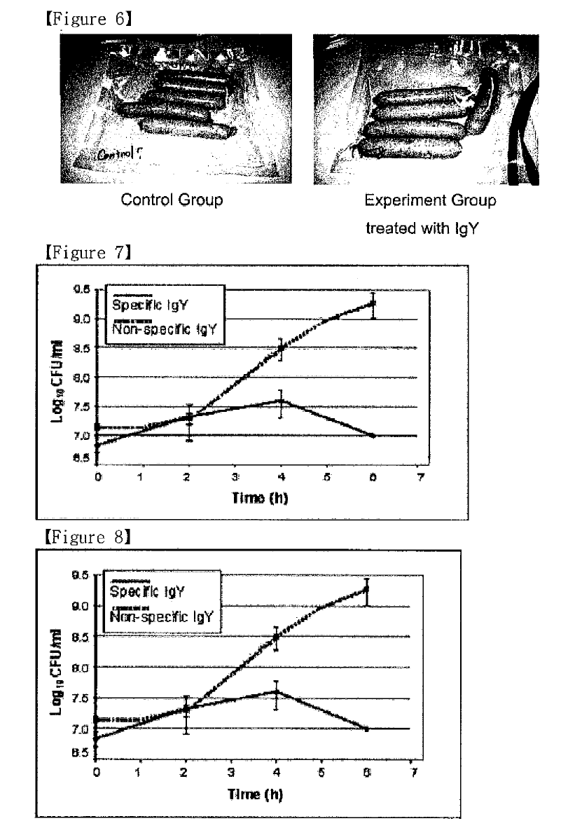

In this Experimental Example, sterilization and growth-inhibitory effects of yolk powder containing IgY with antibacterial activity against 12 pathogens was analyzed by applying it to actual processed meat product. The experiment results show that when Grill Winner sausage was manufactured to contain 0.5% (weight) of yolk powder with IgY, 10¹ of sterilization effect was detected after it was stored for 21 days at the storage temperature of 20° C. (FIG. 4). This sterilization effect is attributed to lowered or lost cell membrane function of bacteria due to deformation of the structure of the cell membrane by binding specific immunoglobulin, IgY, to the cell membrane of the bacteria (FIG. 5). Since IgY has an antibacterial mechanism based on deterioration of bacteria's functions, it is more useful in the aspect of growth-inhibitory function rather than inactivation through sterilization. As processed meant products such as ham or sausage are not entirely pasteurized, the goal of microbial safety is to arrest the growth of pathogens and bacteria down below 10¹˜10². As IgY has a selective growth-inhibitory effect against a specific pathogen, it can be noted that IgY is effective in the aspect of microbial safety. Therefore, it is possible to effectively sterilize and control the growth of target bacteria without affecting taste when yolk powder containing IgY with selective antibacterial ability is added.

### EXPERIMENTAL EXAMPLE 3

### Measurement of Antibacterial Effect of Liquid-Type Yolk Containing 2 Types of IgY

In this Experiment Example, sterilization and growth-inhibitory effects of yolk powder containing IgY with antibacterial activity against particularly two pathogens were analyzed by applying actual processed meat product. The two target pathogens include Listeria and Salmonella that have recently been showing frequent appearances in processed meat food stuff. In this experiment, two types of IgY that showed an effect to these pathogens in the preliminary test were mixed and tested.

The experiment results show that when Grill Winner sausage was manufactured containing 0.5% (weight) of yolk liquid containing the two types of IgY, no significant change was detected in terms of the total number of bacteria after being stored for 14 days at the storage temperature of 15° C. However, in the control group without IgY addition, phlegmatic temperament that indirectly indicates the state of decay was generated, and the package was swollen due to gas generation—whereas, none of these state changes was noticeable in the experimental group, which shows favorable quality. Experimental Example 3 reconfirms the results of Experimental Example 2 on IgY's excellent microbial growth-inhibitory effect.

### EXPERIMENTAL EXAMPLE 4

### Measurement of Antibacterial Effect of IgY of the Present Invention

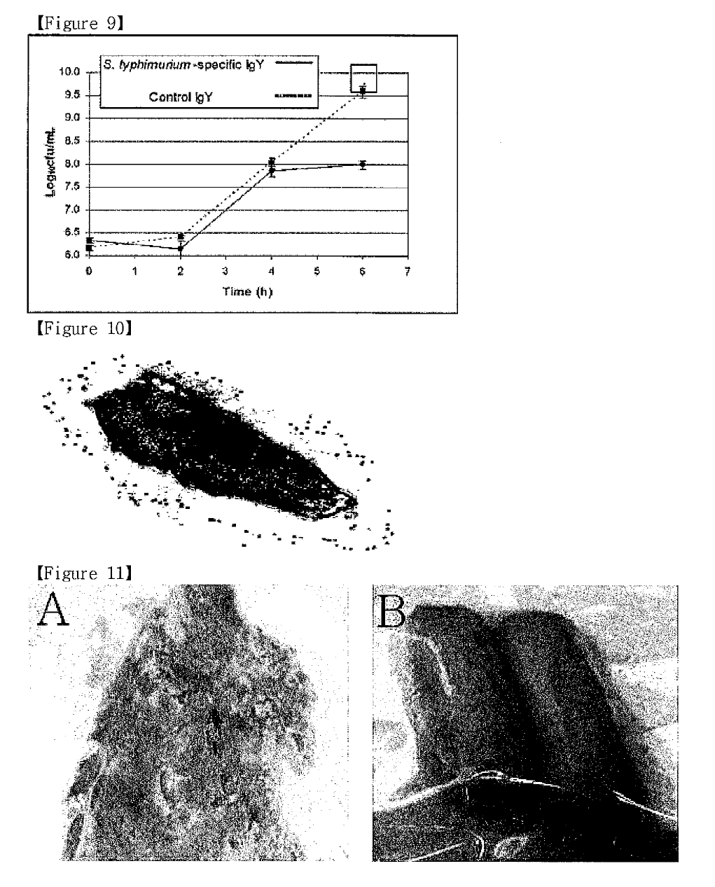

Vaccines for each of 0157:H7 (Escherichia coli 0157:H7), Salmonella enteritidis and Salmonella typhimurium were injected to obtain liquid-type IgY with antibacterial effect (sterilization and growth inhibition) against each of microbes. The liquid-type IgY was lyophilized to be processed into powder-type substance, and then ELISA based on immune enzyme response method was operated to confirm antibacterial effect against each of the specific pathogens including 0157:H7 (Escherichia coli 0157:H7), Salmonella enteritidis, and Salmonella typhimurium. Afterwards, three saline solutions were prepared that contain 10⁶˜10⁷ CFU/ml 0157:H7 (Escherichia coli 0157:H7), Salmonella enteritidis, and Salmonella typhimurium each. Afterwards, 2.34 mg/ml IgY powder against 0157:H7 (Escherichia coli 0157:H7), 0.9 mg/ml IgY powder against Salmonella enteritidis and 0.63 mg/ml IgY powder against Salmonella typhimurium were added in the respective saline solutions. The sterilization and growth-inhibitory effects against the three types of bacteria were observed over time by detecting the decrease in the number of bacteria. Except for the selection of target bacteria, IgY powder manufacturing was conducted in accordance with Example 1. The results showed that in the case of IgY powder against 0157:H7 (Escherichia coli 0157:H7), 10² (CFU/ml) sterilization effect (99% of 0157:H7 sterilized) was observed (FIG. 7). As for Salmonella enteritidis, 10¹ (CFU/ml) sterilization effect was confirmed (90% of Salmonella enteritidis sterilized) (FIG. 8). IgY powder against Salmonella typhimurium showed 10¹ (CFU/ml) sterilization effect (90% of Salmonella typhimurium sterilized) (FIG. 9).

In order to figure out the mechanism of IgY's sterilization or growth inhibition against 0157:H7 (Escherichia coli 0157:H7), IgY treated 0157:H7 (Escherichia coli 0157:H7) was observed through an electron microscope. As seen in FIG. 10, IgY Immunogold particle is attached to the cell membrane of 0157:H7 (Escherichia coli 0157:H7). After approximately four hours, the cell membrane of this pathogen is deformed to cause the pathogen's death or inhibited growth.

The powder-type IgY obtained through lyophilization of IgY water solution separated according to Process 2 of Example 1 was added to Grill Winner sausage. The sausage was manufactured containing 0.05% (weight) of the above mentioned powder type IgY, and was kept in the incubator at the temperature of 35° C. After seven days, the changes in the appearance of the sausage caused by the growth of microbes (pathogens and other microbes deteriorating product quality) were observed and analyzed. The results show that the control group (A) without IgY treatment, the texture was completely broken due to the growth of microbes, as seen in FIG. 11. However, in the experimental group with IgY addition (B), the microbes were sterilized or their growth was inhibited so that the texture of the product remained intact. This confirms the excellent antibacterial effect of IgY of the present invention.

### INDUSTRIAL APPLICABILITY

The composition containing IgY with the antibacterial activation against 12 pathogens of the present invention, designed to sterilize and inhibit the growth of pathogenic bacteria, has an outstanding antimicrobial effect against bacteria that cause food poisoning and decay of meat-based processed food. The composition of the present invention also shows an excellent effect to sterilize and inhibit the growth of pathogenic bacteria at the low temperature, non-thermal treatment condition. Therefore, it is an outstanding invention in the food sterilization industry as it can be commercially-used in various ways as natural sterilizer.

1. A method of manufacturing specific immunoglobulin in yolk comprising the steps:
i) producing egg containing specific immunoglobulin by injecting vaccines against pathogenic bacteria and other microbes that deteriorate product quality to chickens;
ii) sterilizing the yolk separated from the egg at a low temperature; and
iii) adding acidic water solution to the sterilized yolk and centrifuging the resulted mixture.

2. The method of manufacturing specific IgY of claim 1, wherein the low temperature sterilization in step ii) is carried out at 50-75° C. for 15 minutes to 1 hour.

3. The method of manufacturing specific IgY of claim 1, wherein the centrifugation in step iii) is performed after the final acidity is adjusted to between pH 5.2 and pH 5.5 by adding acidic water solution to sterilized yolk.

4. The method of manufacturing specific IgY of claim 1, wherein the pathogenic bacteria and other microbes that deteriorate product quality are Aeromonas hydrophila, Bacillus cereus, Campylobacter jejuni, Clostridium perfringens, 0157:H7 (Escherichia coli 0157:H7), Lactobacillus, Listeria monocytogens, Sacromyces cerevisae, Salmonella enteritidis, Salmonella typhimurium, Staphylococcus aureus and Staphylococcus epidermidis.

5. A specific IgY manufactured by one of methods of manufacturing specific IgY of claim 1 to claim 4.

6. Egg yolk containing specific IgY manufactured by a method comprising the steps:
i) producing egg containing specific immunoglobulin by injecting vaccines against pathogenic bacteria and other microbes that deteriorate product quality to chickens; and
ii) sterilizing the yolk separated from the egg at a low temperature.

7. Egg yolk powder containing specific IgY manufactured by lyophilizing the yolk of claim 6.

8. Food additive composition for sterilization or decay-inhibitory functions of meat-based processed food comprising one or more selected from the group that consists of the specific IgY according to claim 5, the yolk containing the specific IgY according to claim 6 and the yolk powder containing the specific IgY according to claim 7.
